(19) United States
(12) Patent Application Publication
(10) Pub. No.: US 2024/0058294 A1
(43) Pub. Date: Feb. 22, 2024

(54) NOVEL MULTILAYER SUPPOSITORY AGENT

(71) Applicant: INDUSTRY-ACADEMIC COOPERATION FOUNDATION, YONSEI UNIVERSITY, Seoul (KR)

(72) Inventors: Sung-Joo Hwang, Seoul (KR); Seung Hyuk Baik, Seoul (KR); Eun Jung Park, Seoul (KR); Se Hyeon Jeong, Seoul (KR); Intaek Shin, Incheon (KR)

(21) Appl. No.: 17/762,718

(22) PCT Filed: Sep. 23, 2020
(86) PCT No.: PCT/KR2020/012894

§ 371 Date: Mar. 22, 2022

(30) Foreign Application Priority Data
Sep. 23, 2019 (KR) 10-2019-0116848

(51) Int. Cl. A61K 31/282; A61K 9/02; A61K 31/519; A61K 31/513; A61K 47/34; A61K 47/36; A61K 47/10; A61K 9/00; A61M 31/00

(52) U.S. Cl. CPC A61K 31/282; A61K 9/025; A61K 31/519; A61K 31/513; A61K 47/34; A61K 47/36; A61K 47/10; A61K 9/0031; A61M 31/00

(57) ABSTRACT

The present invention relates to a multilayer suppository formulation comprising an outer layer containing a first pharmaceutical ingredient and an inner layer containing a second pharmaceutical ingredient, and a composition comprising the same for prevention or treatment of colorectal cancer. The present invention reproduces the release order and release interval of composite drugs administered sequentially in combination, for example, FOLFOX, in the same manner. Accordingly, the present invention can be used as an effective dosage form that concentrates a pharmacological effect, reduces a side effect, and greatly improves dosing convenience for a patient by locally administering combined drugs which had been co-administered systemically through intravenous injection, in a lesion-specific manner.

### TECHNICAL FIELD

The present invention relates to a novel multilayered suppository formulation including a plurality of pharmacological ingredients.

### TECHNICAL FIELD

Colorectal cancer accounts for about 33% of all types of cancer, is the second most common cancer in Korea after stomach cancer, and annually occurs in 26,978 patients per 100,000 people. Colorectal cancer is classified into colon cancer and rectal cancer depending on the area of the site of occurrence, wherein the rectal cancer is defined as cancer occurring from the anus to about 15 cm therefrom, and various treatment methods are being performed depending on the location of the cancer.

Particularly, since mid and low rectal cancer is located very close to the anus, and the rectum invaded by the cancer must be excised and anastomized in the narrow pelvic cavity during surgery, the difficulty of the surgery is very high. In addition, when rectal cancer invades the anal sphincter due to progression thereof, the anus cannot be preserved during surgery, so there is a risk of living with a permanent stoma for the rest of one's life. In consideration of the decrease in the quality of a patient's life and a therapeutic effect after surgery, currently, for the treatment of advanced mid and low rectal cancer, which corresponds to stages 2 and 3, a treatment that can preserve the anus as much as possible and improve a survival rate through surgery conducted after the stage of cancer is lowered (down-staging) by reducing the degree of tissue invasion of tumors by preoperative concurrent chemoradiotherapy is being recommended.

Preoperative concurrent chemoradiotherapy is performed by irradiating a total of 50.4 Gy of radiation around the rectum for about 5 weeks before surgery and concurrently co-administering systemic anticancer agents, such as 5-fluorouracil (5-FU) and leucovorin, at week 1 and week 5. Then, after a resting phase for about 6 to 8 weeks to downsize a tumor, rectal cancer surgery is performed (FIG. 1). However, since chemotherapy causes systemic side effects by destroying normal cells of the heart, kidney, lung, and nervous system as well as the bone marrow where division and proliferation actively occur, the mucous membrane from the oral cavity to the anus and hair follicles, as well as cancer cells, it is important to improve and optimize an administration route and a dosage form in order to maximally protect normal cells by preventing or minimizing such side effects and enhance the convenience of administration to a patient.

Numerous papers and patent documents are referred to throughout the specification, and their citations are indicated. The disclosed contents of the cited papers and patent documents are incorporated herein by reference in their entirety, so that the level of the technical field to which the present invention belongs and the content of the present invention will be more clearly described.

### DISCLOSURE

### Technical Problem

The inventors made intensive research efforts to develop an effective dosage form that can more concentrate a pharmacological effect and reduce side effects by locally administering combined drugs which were conventionally co-administered through intravenous injection in a lesion-specific manner. As a result, the inventors found that, when a multilayered suppository formulation, which includes a first pharmacological ingredient primarily administered and a second pharmacological ingredient secondarily administered, respectively, in an outer layer and an inner layer, is rectally administered through intravenous injection, the first pharmacological ingredient and the second pharmacological ingredient are sequentially released and the therapeutic effect of the suppository formulation is intensively exerted on the rectum, which is a lesion. Moreover, when the suppository formulation is systemically administered, the side effect range may be minimized. Thus, the present invention was completed.

Therefore, the present invention is directed to providing a multilayered suppository formulation, which includes an outer layer including a first pharmacological ingredient and an inner layer including a second pharmacological ingredient.

The present invention is also directed to providing a composition for preventing or treating colorectal cancer, which includes the suppository formulation described above.

The present invention is also directed to providing a method of preparing a multilayered suppository formulation having a core-shell structure.

Other objects and advantages of the present invention will become clearer by the following detailed description of the invention, claims and drawings.

### Technical Solution

According to one aspect of the present invention, a multilayered suppository formulation, which includes a first layer including a first pharmacological ingredient and a second layer including a second pharmacological ingredient, is provided.

The inventors made intensive research efforts to develop an effective dosage form that can more concentrate a pharmacological effect and reduce side effects by locally administering combined drugs which were conventionally co-administered through intravenously injection in a lesion-specific manner. As a result, when a multilayered suppository formulation, which includes a first pharmacological ingredient that is first administered and a second pharmacological ingredient that is administered later, respectively, in an outer layer and an inner layer, is rectally administered through intravenous injection, the inventors found that, although the first pharmacological ingredient and the second pharmacological ingredient are sequentially released, the therapeutic effect can be intensively exerted on the rectum, which is a lesion, and when the suppository formulation is systemically administered, the side effect range can be minimized.

The term “multilayered formulation” used herein means a solid formulation composed of a plurality of layers consisting of different ingredients and having distinct boundaries. When the multilayered formulation is composed of a plurality of layers, it has a laminated multilayer, core-shell multilayer, or a combination thereof without limitation.

According to an exemplary embodiment of the present invention, the multilayered formulation has a core-shell structure in which the first layer is an outer layer and the second layer is an inner layer.

The term “outer layer” used herein refers to a layer surrounding the inner layer of the core-shell structure and farther from the core than the inner layer, and the term “inner layer” used herein refers to a layer closer to the core than the outer layer.

According to the present invention, the multilayered formulation of the present invention is designed for combined drugs to sequentially act by first releasing a drug contained in the outer layer exposed to a body temperature environment after administration, and releasing a drug contained in the inner layer exposed following the completion of dissolution of the outer layer.

The formation of a core-shell structure may be accomplished by various processes known in the art, and for example, may be accomplished by injecting a molten inner layer base into an outer layer base before the outer layer is completely solidified after the outer layer is formed through melting, or coating a formulation composed of only an inner layer with the outer layer.

The term “coating” used herein refers to forming a new layer having a uniform thickness by modifying a specific material on a target surface. The target surface and the coating material may be modified through various chemical bonds such as ionic bonds, covalent bonds, and hydrogen bonds. In the present invention, when the inner layer is coated with the outer layer, the outer layer may completely cover the surface of the inner layer to form an enclosed layer, or may form a partially-enclosed layer.

The term “first pharmacological ingredient” used herein refers to a drug that is included in the outer layer and released earlier than the second pharmacological ingredient included in the inner layer. Therefore, the first pharmacological ingredient is not necessarily a first-released pharmacological ingredient, and may further include an outermost layer including a drug located more outward than the first pharmacological ingredient and released earlier than the first pharmacological ingredient.

The term “second pharmacological ingredient” used herein refers to a drug contained in the inner layer and released later than the first pharmacological ingredient contained in the outer layer. Therefore, the second pharmacological ingredient is not necessarily the finally-released pharmacological ingredient, and may further include a core layer located closer to the core than the second pharmacological ingredient and containing a drug released following the second pharmacological ingredient.

According to an exemplary embodiment of the present invention, the first pharmacological ingredient of the present invention includes one or more selected from the group consisting of oxaliplatin, folinic acid, and a pharmaceutically acceptable salt thereof.

The term “pharmaceutically acceptable salt” used herein refers to a salt derived from a pharmaceutically acceptable inorganic acid, organic acid or base. Examples of appropriate acids may include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, trifluoroacetic acid, citric acid, methanesulfonic acid, formic acid, benzoic acid, malonic acid, naphthalene-2-sulfonic acid, and benzenesulfonic acid. Examples of appropriate salts derived from bases may include an alkali metal such as sodium, an alkaline earth metal such as magnesium, and ammonium.

According to an exemplary embodiment of the present invention, the second pharmacological ingredient of the present invention is 5-FU, Tegafur, or a pharmaceutically acceptable salt thereof.

According to an embodiment of the present invention, a suppository formulation including oxaliplatin, folinic acid or a combination thereof as a first pharmacological ingredient and 5-FU or Tegafur as a second pharmacological ingredient has the same pharmacological ingredient and dosing sequence as FOLFOX, which is a combination of chemotherapeutic agents for colorectal cancer.

According to an exemplary embodiment of the present invention, the first layer including the first pharmacological ingredient further includes a rapid-release inducer.

The multilayered formulation of the present invention may optimize the time interval for a pharmacological ingredient to act in the rectum by inducing immediate release from an outer layer and sustained release from an inner layer through adjustment of the release rate of each layer, in addition to sequential release according to the dissolution order of the outer and inner layers.

The term “rapid-release inducer” used herein refers to a natural or synthetic polymer that aids the rapid release of a pharmacological ingredient in a formulation to allow a rapid pharmacological effect to be exhibited. As the rapid-release inducer of the present invention, various ingredients known in the art to induce the rapid release of a pharmacological ingredient may be used, and for example, rapid release may be induced by mixing and encapsulating polyethylene glycol (PEG) and the first pharmacological ingredient in the outer layer. Since PEG has a high melting point (e.g., the melting point of PEG400 is 55° C.), but has a mechanism of being dissolved by the body fluid of the rectum, the rapid release of the first pharmacological ingredient may be induced. Specifically, rapid release may be induced by the use of PEG4000, PEG400, or a mixture thereof, more specifically, a mixture of PEG4000 and PEG400 in a ratio of 3:1 to 5:1 (w/w), and most specifically, a mixture of PEG4000 and PEG400 in a ratio of 4 to 1 (w/w).

According to an exemplary embodiment of the present invention, the second layer including the second pharmacological ingredient further includes a sustained-release inducer. The term “sustained-release inducer” used herein refers to a natural or synthetic polymer aiding in the slow release of a pharmacological ingredient in a formulation to allow the pharmacological effect to be sustained for a desired period of time. As the sustained-release inducer of the present invention, various ingredients known in the art to have an activity of delaying the release of a pharmacological ingredient may be used, and representatively, a mucoadhesive agent may be used. Therefore, for example, the sustained release of the second pharmacological ingredient may be induced by mixing and encapsulating a mucoadhesive agent and the second pharmacological ingredient in the inner layer.

The term “mucoadhesive agent” used herein refers to a synthetic or natural polymer which forms a covalent bond, an ionic bond, and a hydrogen bond with a mucus layer covering the mucosal epithelial surface in mucosal tissues such as the nasal cavity, the esophagus, the stomach, the small intestine, and the rectum.

The sustained-release inducer (or mucoadhesive agent) available in the present invention may be a poloxamer, Carbopol, hydroxypropyl methylcellulose (HPMC), sodium alginate, chitosan, or λ-carrageenan, but the present invention is not limited thereto. Specifically, the sustained-release inducer is a poloxamer, λ-carrageenan, Carbopol, or a combination thereof. More specifically, the poloxamer is poloxamer 188.

According to the present invention, the multilayered suppository according to the present invention may be reproduced not only to have the same pharmacological ingredients and the same dosing order as those of FOLFOX but also the same or similar time interval for each pharmacological ingredient to act, by inducing rapid release by mixing PEG with the first pharmacological ingredient (e. g, oxaliplatin and folinic acid) and inducing sustained release by mixing a mucoadhesive agent with the second pharmacological ingredient (e.g., 5-FU).

According to an exemplary embodiment of the present invention, the second layer including the second pharmacological ingredient further includes a plasticizer. The component “plasticizer” of the present invention lowers a melting point to a level similar to body temperature in order to prevent a decrease in release efficiency in the body due to the high melting point of the sustained-release inducer or mucoadhesive agent included in the second layer.

More specifically, the plasticizer used in the present invention is propylene glycol. For example, when poloxamer 188 having a melting point of 55° C. is used as a mucoadhesive agent, propylene glycol may be added thereto as a plasticizer in a ratio of 6:4 to 4:6 (w:w), so that the melting point may be adjusted to a level similar to body temperature.

According to one embodiment of the present invention, the suppository formulation of the present invention may further include an outermost layer surrounding the outer layer, which includes a mucoadhesive agent.

The term “outermost layer” used herein refers to a layer in which one surface is in contact with another layer in the multilayered formulation, and the other surface faces the outside.

The term “mucoadhesive agent” that can be used in the preset invention has been described above in detail, so the description will be omitted to avoid excessive duplication. According to the present invention, when the suppository formulation of the present invention further includes the outermost layer including a mucoadhesive agent, adsorption to the rectum wall may be promoted after rectal administration to allow a pharmacological ingredient to be rapidly absorbed into tissue and lymph nodes through blood vessels developed around the rectum, thereby increasing a local treatment effect.

According to one embodiment of the present invention, the suppository formulation of the present invention further includes a core layer in the inner layer, and the core layer includes a mucosal protective agent.

The term “core layer” used herein refers to an innermost layer with only one surface in contact with another layer in the multilayered formulation.

The “mucosal protective agent” used herein refers to a material that is applied to mucosal tissues such as the nasal cavity, the esophagus, the stomach, the small intestine, and the rectum, thereby forming a physical or chemical barrier against surrounding stimulus such as an acid, corrosive damage, or drug irritation. The mucosal protective agent of the present invention is finally applied to the rectal wall after the suppository formulation of the present invention is administered and all pharmacological ingredients are released, thereby providing a protective film.

The mucosal protective agent of the present invention may be any synthetic or natural polymer that has a mucoadhesive property without limitation and selected from the group consisting of, for example, sucralfate, misoprostol and Carbopol, and is more specifically Carbopol.

According to another aspect of the present invention, the present invention provides a composition for preventing or treating colorectal cancer, which includes the suppository formulation of the present invention described above.

Since the configuration of the suppository formulation used in the present invention has already been described above, the description thereof will be omitted to avoid excessive duplication.

The term “prevention” used herein refers to inhibiting the occurrence of a disease or disorder in a subject who has not been diagnosed with a disease or disorder, but is likely to have the disease or disorder.

The term “treatment” used herein refers to (a) inhibiting the development of a disease, a disorder, or a symptom; (b) alleviating a disease, a disorder, or a symptom; or (c) eliminating a disease, a disorder, or a symptom. As a plurality of pharmacological ingredients are sequentially released from the rectum at intervals of time, the formulation of the present invention serves to inhibit the development of a symptom of a disease that occurs in the rectum as a lesion site, or where a pharmacological ingredient can be effectively delivered through rectal administration, or to eliminate or reduce the symptom. Therefore, the composition of the present invention may be used as a therapeutic composition by itself, or may be administered together with another pharmacological ingredient to be applied as a therapeutic adjuvant for the disease. Therefore, the term “treatment” or “therapeutic agent” used herein means therapeutic aid or a therapeutic adjuvant.

The term “administration” or “administer” used herein means that a therapeutically effective amount of the formulation of the present invention is directly administered to a subject, so that a pharmacological ingredient included in the formulation of the present invention is formed in a subject's body at substantially the same amount.

The “therapeutically effective amount” of the formulation refers to a dose of the formulation in which the content of a pharmacological ingredient in the formulation is sufficient to provide a therapeutic or preventive effect to a subject, and includes a “prophylactically effective amount.” The term “subject” used herein is a human, a mouse, a rat, a guinea pig, a dog, a cat, a horse, a cow, a pig, a monkey, a chimpanzee, a baboon, or a rhesus monkey without limitation. Specifically, the subject of the present invention is a human.

As a pharmaceutical composition, the formulation of the present invention includes a pharmaceutically acceptable carrier. The pharmaceutically acceptable carrier included in the pharmaceutical composition of the present invention is conventionally used in formulation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, methyl cellulose, methylhydroxybenzoate, propylhydroxybenzoate, talc, magnesium stearate, mineral oil, saline, phosphate buffered saline (PBS), and a medium, but the present invention is not limited thereto.

The formulation of the present invention may further include a lubricant, a wetting agent, an emulsifier, a suspending agent, or a preservative, in addition to the above-described ingredients. Suitable pharmaceutically acceptable carriers and agents are disclosed in the Remington's Pharmaceutical Sciences (19th ed., 1995) in detail.

A suitable dosage of the formulation of the present invention may be variously determined by parameters such as a formulation method, a patient's age, weight, sex, morbidity, food, administration time, excretion rate, and responsiveness.

According to an exemplary embodiment of the present invention, colorectal cancer treated by the composition of the present invention is rectal cancer, and more specifically, mid and low rectal cancer.

The term “rectal cancer” used herein refers to a type of colorectal cancer in which a distance from the anus to the lower edge of a tumor is within 15 cm. The term “mid and low rectal cancer” used herein refers to a type of rectal cancer in which a distance from the anus to the lower edge of a tumor is within 9 cm.

In the case of mid and low rectal cancer, the lesion is located close to the anus so that the location of the cancer can be palpated with a finger, and thus when the suppository formulation of the present invention is directly administered to the anal cavity, a pharmacological ingredient may be rapidly absorbed into the surrounding tissues and lymph nodes through the blood vessels developed around the rectum, thereby enhancing the effect of local treatment.

According to still another aspect of the present invention, the present invention relates to a method of preventing or treating colorectal cancer, which includes administering the suppository formulation of the present invention into a subject.

Since the suppository formulation used in the present invention and the colorectal cancer to be prevented or treated using the same have already been described above, the description thereof will be omitted to avoid excessive overlapping.

According to yet another aspect of the present invention, the present invention provides a method of preparing a multilayered suppository formulation having a core-shell structure, which includes the following steps:
(a) dissolving a first ingredient including one or more pharmacological ingredients selected from the group consisting of oxaliplatin, folinic acid and a pharmaceutically acceptable salt thereof;
(b) injecting the first ingredient dissolved in Step (a) into a mold;
(c) dissolving a second ingredient including 5-FU, Tegafur or a pharmaceutically acceptable salt thereof and solidifying the second ingredient; and
(d) injecting the second ingredient solidified in Step (c) into the center of the mold into which the first ingredient is injected before the first ingredient injected in Step (b) is completely solidified.

Since the multilayered formulation to be prepared in the present invention and the colorectal cancer to be treated using the same have been already described, the description thereof will be omitted to avoid excessive overlapping.

The “dissolution” used herein refers to forming a liquid or gel by heating a solid first or second ingredient to provide flowability, which facilitates molding through a molding technique. The dissolution may be performed by reaching a melting point without affecting the pharmacological activity of each ingredient, and may be performed by, for example, heating in water at 50 to 70° C., and specifically, 55 to 65° C.

Afterward, the first ingredient forming an outer layer is injected into the mold having the form of a suppository, and then the second ingredient forming an inner layer is injected into the center of the first ingredient before complete solidification. Here, the second ingredient is injected 60 to 180 seconds, more specifically, 100 to 140 seconds, and most specifically, 110 to 130 seconds after the injection of the first ingredient.

Steps (c), and (a) and (b) may be performed sequentially, simultaneously, or in reverse order.

### Advantageous Effects

The characteristics and advantages of the present invention are summarized as follows:

(a) The present invention provides a multilayered suppository formulation which includes an outer layer including a first pharmacological ingredient and an inner layer including a second pharmacological ingredient, and a composition for preventing or treating colorectal cancer using the same.

(b) The present invention reproduces the release order and release interval of combined drugs, for example, FOLFOX, which are sequentially co-administered.

(c) The present invention can be used as an effective dosage form that concentrates a pharmacological effect, reduces a side effect, and greatly improves dosing convenience for a patient by locally administering combined drugs which had been co-administered systemically through intravenous injection, in a lesion-specific manner.

### DESCRIPTION OF DRAWINGS

FIG. 1 shows a preoperative concurrent chemoradiotherapy procedure for mid and low rectal cancer.

FIG. 2 is a schematic diagram of a core-shell structure of an anticancer agent of the present invention, showing an exemplary embodiment that an outermost layer is additionally coated with a mucoadhesive layer, and a mucosal protective agent is additionally inserted into an innermost layer.

FIG. 3 shows an image of a core-shell suppository manufactured in the present invention.

FIG. 4 shows release rates of oxaliplatin and 5-FU over time in the core-shell suppository prepared in the present invention.

FIG. 5 shows an image of a laminated double-layer suppository manufactured in the present invention.

FIG. 6 shows release rates of oxaliplatin and 5-FU over time in the laminated double-layer suppository manufactured in the present invention.

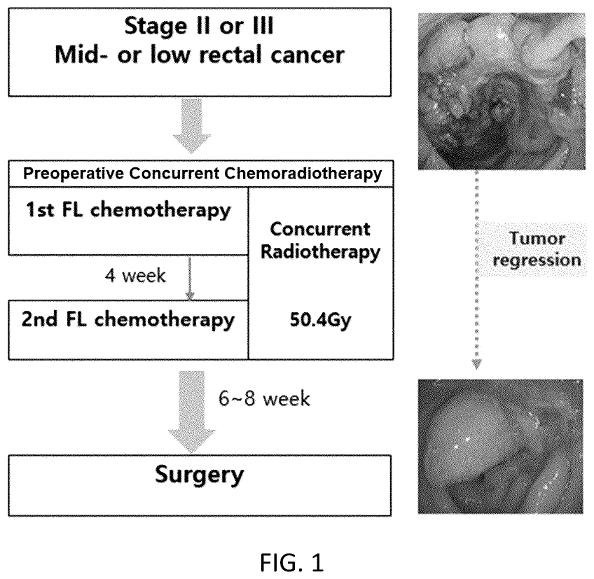

### MODES OF THE INVENTION

Hereinafter, the present invention will be described in further detail with reference to examples. The examples are merely provided to more specifically explain the present invention, and it will be obvious to those of ordinary skill in the art that the scope of the present invention is not limited to the examples according to the gist of the present invention.

### EXAMPLES

### Experimental Method

Production of Core-Shell Suppository

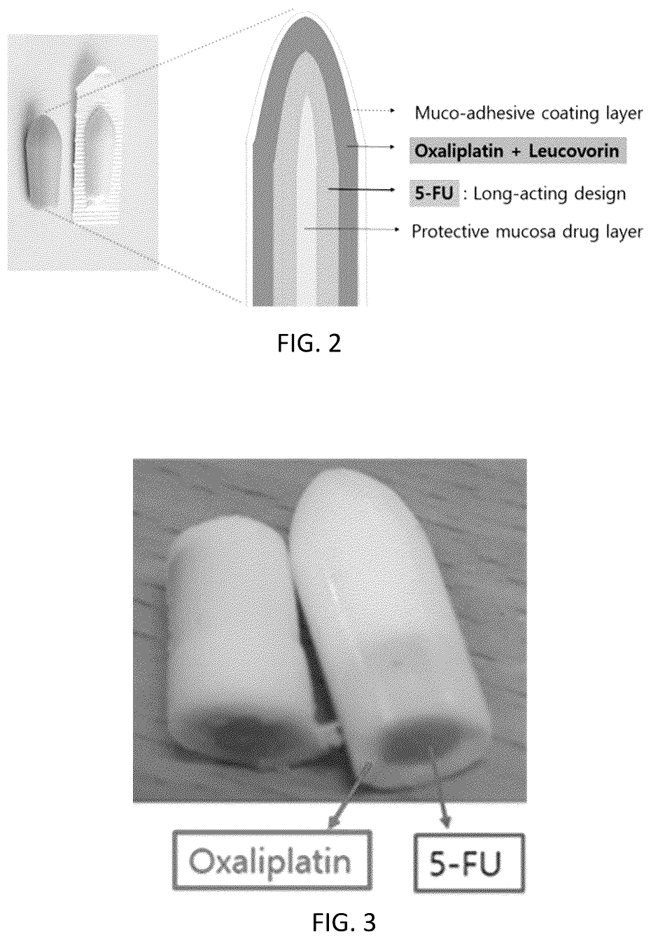

The inventors produced a core-shell structure having the form of an inner-outer layer as a first form of a suppository including two types of pharmacological ingredients with different release characteristics. The present invention intended to implement a composite anticancer agent FOLFOX, which includes a combination of oxaliplatin, folinic acid and 5-FU, in the form of a suppository, and thus the outer layer consisted of a mixture of oxaliplatin and folinic acid (leucovorin) and the inner layer consisted of 5-FU, so that the oxaliplatin+folinic acid of the outer layer was exposed due to a temperature change caused by a body temperature after rectal administration and absorbed into cancer tissue, and when the inner layer is exposed as the release of the outer layer was terminated, the 5-FU formulation was released to be absorbed into the cancer and surrounding tissues (FIG. 2).

For the production of the core-shell suppository, melting was used. As the core (inner layer) suppository, 80 mg of 5-FU (Tokyo Chemical Industry Co., Ltd, Tokyo, Japan) was used, for a sustained-release effect, Carbopol (Carbopol® Ultrez 20, Lubrizol Advanced Materials, Inc., Cleveland, USA) and λ-carrageenan (FMC, Philadelphia, USA) were added, and as a filling mold, a cylinder for a suppository with a size of 1.5 cm×0.5 cm was used. The dosage form of the core suppository was produced so that a mass ratio of Foloxamer 188 (Sigma-Aldrich, Saint Louis, USA): propylene glycol (Daejung Chemical & METALS Co., Ltd, Siheung, Korea): Carbopol: λ-carrageenan: 5-FU is 52.5:32.5:5:5:5. The mixture used in the construction of the core suppository was heated in water at 60° C. for 10 minutes and completely melted, and then filled in a suppository mold without including air. The filled suppository mold was refrigerated at less than 4° C. for 6 hours, an inlet was eliminated, a solidified suppository was taken out of the mold, and the resulting suppository was stored in the refrigerator.

Following completely solidifying the core suppository, a shell (outer layer) suppository was produced. For the shell suppository, 30 mg of oxaliplatin (Boryung Pharmaceutical, Jongno, Korea) was added, and as a filling mold, an aluminum suppository mold having a size of 3.1 cm×1 cm was used. A dosage form of the shell suppository was produced such that a mass ratio of PEG4000 (SAMCHUN Chemicals, Seoul, Korea):PEG400 (SAMCHUN Chemicals, Seoul, Korea):oxaliplatin is 79:19:2. The mixture used in the production of the shell suppository was heated in water at 70° C. for 10 minutes to completely melt a base, and then poured into the suppository mold. After the suppository mold was filled, the edge of the shell was slightly solidified at room temperature for two minutes. After two minutes, the core suppository was inserted into the central region of the shell. After the core suppository was completely inserted into the shell suppository, it was refrigerated at less than 4° C. for 6 hours, and then the resulting suppository was taken out of the mold.

Production of Double-Layered Suppository

The inventors produced a double-layered suppository as a second type of the suppository including two pharmacological ingredients and having different release characteristics, and 80 mg of 5-FU was added to the first layer, and 30 mg of oxaliplatin was added to the second layer. The first layer of the double-layered suppository was produced to have a mass ratio of 52.5:32.5:5:5:5 (Poloxamer 188:propylene glycol:Carbopol:λ-carrageenan:5-FU), and the second layer thereof was produced to have a mass ratio of 79:19:2 (PEG4000: PEG400: oxaliplatin). The production of the double-layered suppository was performed in the same manner as in the production of the core-shell suppository. Briefly, the base of the first suppository was heated in water at 60° C. for 10 minutes until the base was completely melted, and then Carbopol, λ-carrageenan and 5-FU were slowly added and mixed. The resulting mixture was uniformly mixed and then poured into a suppository mold, refrigerated at less than 4° C. for 6 hours to solidify the suppository. After the first-layer suppository was completely solidified, a second-layer suppository was produced. A base of the second-layer suppository was heated in water at 70° C. for 10 minutes until the base was completely melted, and then oxaliplatin was slowly added and mixed. The resulting mixture was uniformly mixed, and the second-layer suppository was poured into the suppository mold containing the first-layer suppository to fill. After the suppository mold was filled with the second-layer suppository, it was refrigerated at less than 4° C. for 6 hours, and then the resulting suppository was taken out of the mold.

FOLFOX Suppository Release Test

A release test was performed in the same way for the core-shell suppository and the double-layered suppository. In summary, the release test for the suppository was performed using a dissolution tester (DISTEK dissolution tester 2500, Montreal, Canada), and to prevent floating of the suppository, the test was performed according to the USP 1 basket method. Conditions for the test were adjusted to 37° C. and 50 rpm, and 500 ml of phosphate-buffered saline with pH 4.4 was used as a release test solution, so that the pH was adjusted to be similar to the pH environment around rectal cancer tissue. Sampling time points were set at 5, 10, 15, 30, 60, 120, 240, 360, 480, 600 and 720 minutes, and the experiment was carried out for up to12 hours. Samples per each time point were filtered through a 0.45-μm nylon filter, and analyzed through high performance liquid chromatography (HPLC) of the Agilent 1200 series system (Agilent Technologies, Santa Clara, Calif, USA). For oxaliplatin analysis, 0.005M sodium 1-heptanesulfonate: MeOH [70:30] was used as a mobile phase, and injected into a 5-μm C18 column (Waters) with a size of 300 mm×3.9 mm at a flow rate of 1 ml/min, and a retention time was 1.87 minutes. Oxaliplatin was detected using a 254-nm UV detector (Agilent Technologies). For 5-FU analysis, 0.005 M KH₂PO₄: MeOH [96:4] was used as a stationary phase, and injected into a 5-μm Phenomenex column with a size of 150 mm×4.6 mm at a flow rate of 1 ml/min, and a retention time was 6.20 minutes. 5-FU was also detected using a 254-nm UV detector (Agilent Technologies).

### Experimental Results

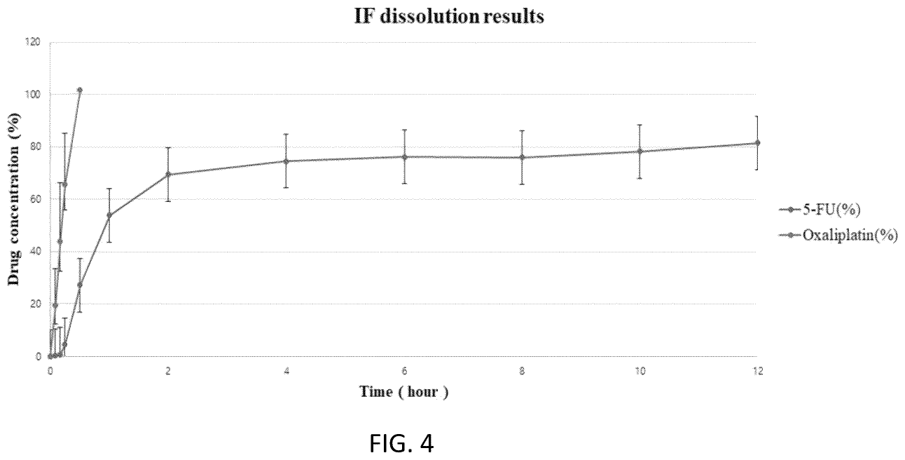

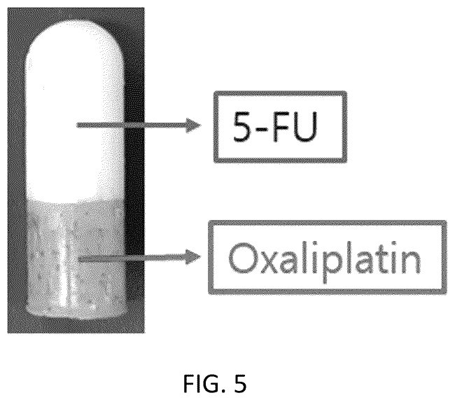

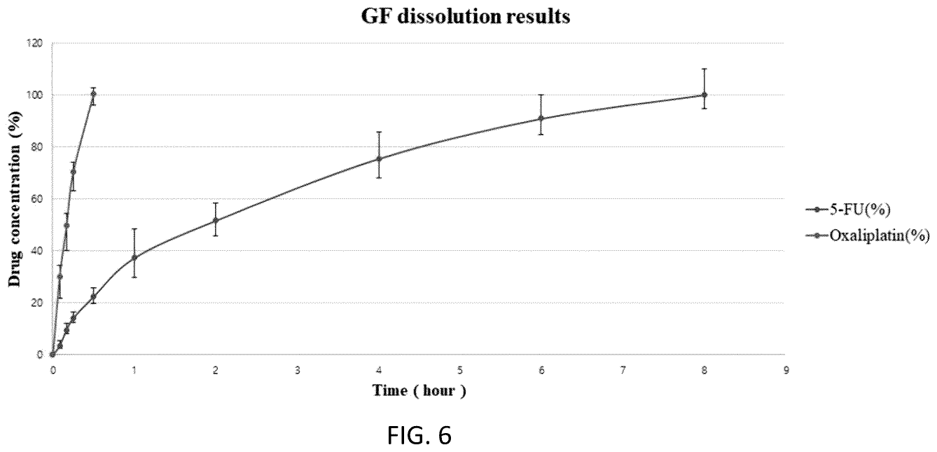

The release patterns of the pharmacological ingredients in the core-shell suppository and the double-layered suppository are shown in FIGS. 4 and 6, respectively. In the core-shell suppository, 5-FU constituting the inner layer (core) suppository was not released until 10 minutes passed, but 69% was released after 2 hours and 80% after 12 hours. For oxaliplatin constituting the inner layer (shell) suppository, 19% and 43% were released at 5 minutes and 10 minutes, respectively, and 100% release was completed within 30 minutes. Therefore, it was found that the moment when the shell suppository was dissolved and the surface of the core suppository was exposed was 10 minutes, indicating that each drug was sequentially released according to a desired order (FIG. 4). In the double-layered suppository, the oxaliplatin of the second layer was completely released within 30 minutes, the 5-FU of the first layer was completely released at 8 hours. Therefore, it can be found that the release patterns of the first and second layer dosage forms designed as a sustained-release type and a rapid-release type are well realized, indicating that a desired drug is sequentially released according to a desired order (FIG. 6). Since the core suppository in the core-shell suppository has a larger release surface area than the first layer of the double-layered suppository, release proceeded faster.

As above, as specific parts of the specification have been described in detail, although it is clear to those skilled in the art that this specific technique is merely a preferred embodiment, the scope of the specification is not limited thereto. Thus, the substantial scope of the specification will be defined by the accompanying claims and their equivalents.

1. A multilayered suppository formulation, comprising:
a first layer comprising a first pharmacological ingredient and a second layer comprising a second pharmacological ingredient.

2. The suppository formulation of claim 1, wherein the multilayered formulation has a core-shell structure in which the first layer is an outer layer and the second layer is an inner layer.

3. The suppository formulation of claim 1, wherein the first pharmacological ingredient is one or more selected from the group consisting of oxaliplatin, folinic acid, and a pharmaceutically acceptable salt thereof.

4. The suppository formulation of claim 1, wherein the second pharmacological ingredient is fluorouracil (5-FU), Tegafur or a pharmaceutically acceptable salt thereof.

5. The suppository formulation of claim 1, wherein the first layer comprising the first pharmacological ingredient further comprises a rapid-release inducer.

6. The suppository formulation of claim 5, wherein the rapid-release inducer is polyethylene glycol.

7. The suppository formulation of claim 6, wherein the polyethylene glycol is selected from the group consisting of PEG400, PEG4000, and a combination thereof.

8. The suppository formulation of claim 1, wherein the second layer comprising the second pharmacological ingredient further comprises a sustained-release inducer.

9. The suppository formulation of claim 8, wherein the sustained-release inducer is one or more selected from the group consisting of a poloxamer, Carbopol, hydroxypropyl methylcellulose (HPMC), sodium alginate, chitosan, and k-carrageenan.

10. The suppository formulation of claim 9, wherein the sustained-release inducer is a poloxamer, Carbopol and k-carrageenan.

11. The suppository formulation of claim 8, wherein the second layer comprising the second pharmacological ingredient further comprises a plasticizer.

12. The suppository formulation of claim 11, wherein the plasticizer is propylene glycol.

13. A method for preventing or treating colorectal cancer, comprising administering the suppository formulation of claim 1 to a subject in need thereof.

14. The method of claim 13, wherein the colorectal cancer is rectal cancer.

15. The method of claim 14, wherein the rectal cancer is mid and low rectal cancer.

16. A method of preparing a multilayered suppository formulation having a core-shell structure, comprising:
(a) dissolving a first ingredient including one or more pharmacological ingredients selected from the group consisting of oxaliplatin, folinic acid and a pharmaceutically acceptable salt thereof;
(b) injecting the first ingredient dissolved in Step (a) into a mold;
(c) dissolving a second ingredient including 5-FU, Tegafur or a pharmaceutically acceptable salt thereof and solidifying the second ingredient; and
(d) injecting the second ingredient solidified in Step (c) into the center of the mold into which the first ingredient is injected before the first ingredient injected in Step (b) is completely solidified.

17. The method of claim 16, wherein the suppository formulation is a suppository formulation for preventing or treating mid and low rectal cancer.
